(19) United States
(12) Patent Application Publication
(10) Pub. No.: US 2026/0053459 A1
(43) Pub. Date: Feb. 26, 2026

(54) CONTROL DEVICE, CONTROL METHOD, AND CONTROL PROCESSING PROGRAM

(71) Applicant: FUJIFILM Corporation, Tokyo (JP)

(72) Inventor: Masato NAKAYAMA, Tokyo (JP)

(73) Assignee: FUJIFILM Corporation, Tokyo (JP)

(21) Appl. No.: 19/303,325

(22) Filed: Aug. 18, 2025

(30) Foreign Application Priority Data
Aug. 21, 2024 (JP) 2024-140095

(51) Int. Cl. A61B 6/00; A61B 6/03; A61B 6/42

(52) U.S. Cl. CPC A61B 6/545; A61B 6/032; A61B 6/4241

(57) ABSTRACT

A control device used in a CT scan that acquires a plurality of pieces of projection data of a subject by a photon counting type radiation detector, the control device includes at least one processor configured to specify a scan region and an examination purpose of the subject, acquire a scan parameter based on the specified scan region and examination purpose, and present the acquired scan parameter as part of a scan condition.

### CROSS-REFERENCE TO RELATED APPLICATION

This application claims priority from Japanese Patent Application No. 2024-140095, filed Aug. 21, 2024, the disclosure of which is incorporated herein by reference in its entirety.

### BACKGROUND OF THE INVENTION

### Technical Field

The present disclosure relates to a control device, a control method, and a non-transitory computer-readable storage medium storing a control processing program.

### Related Art

As a computed tomography (CT) apparatus that captures a medical image, a photon counting CT that detects an X-ray photon and acquires energy information thereof is known (for example, refer to JP2022-57301A).

Incidentally, in the photon counting CT, it is equivalent to being able to switch and use CT apparatuses having different features, and simply considering this, the number of examination protocols is multiplied by the number of measurement modes. Therefore, it is not practical to individually set parameters for the protocol considered necessary for an examination as in the related art and to register the parameters in association with the scan region.

### SUMMARY

The present disclosure has been made in consideration of the above circumstances, and an object of the present disclosure is to provide a control device, a control method, and a non-transitory computer-readable storage medium storing a control processing program capable of easily setting a scan parameter even in a case where the number of protocols increases in accordance with the number of measurement modes in the photon counting CT.

In order to achieve the above object, according to a first aspect of the present disclosure, there is provided a control device used in CT scan that acquires a plurality of pieces of projection data of a subject by a photon counting type radiation detector, the control device including at least one processor configured to specify a scan region and an examination purpose of the subject, acquire a scan parameter based on the specified scan region and examination purpose, and present the acquired scan parameter as part of a scan condition.

According to a second aspect, in the control device according to the first aspect, the scan parameter may be associated with at least one of the scan region or the examination purpose based on a rule.

According to a third aspect, in the control device according to the second aspect, the scan parameter may include a plurality of types, and the processor may be configured to acquire a first scan parameter in which a parameter value is associated based on a first rule corresponding to the scan region and a second scan parameter in which a parameter value is associated based on a second rule corresponding to the examination purpose.

According to a fourth aspect, in the control device according to the third aspect, the processor may be configured to present the second scan parameter as part of the scan condition, in a case where a type of the first scan parameter and a type of the second scan parameter are the same.

According to a fifth aspect, in the control device according to the third aspect, the second scan parameter may include at least one of a measurement mode or a focal size.

According to a sixth aspect, in the control device according to the first aspect, the processor may be configured to specify a system state for the CT scan, and acquire the scan parameter further based on the specified system state.

In order to achieve the above object, according to a seventh aspect of the present disclosure, there is provided a control method executed by at least one processor included in a control device used in CT scan that acquires a plurality of pieces of projection data of a subject by a photon counting type radiation detector, the control method including specifying a scan region and an examination purpose of the subject, acquiring a scan parameter based on the specified scan region and examination purpose, and presenting the acquired scan parameter as part of a scan condition.

In order to achieve the above object, according to an eighth aspect of the present disclosure, there is provided a non-transitory computer-readable storage medium storing a control processing program for causing at least one processor included in a control device used in CT scan that acquires a plurality of pieces of projection data of a subject by a photon counting type radiation detector to execute processing including specifying a scan region and an examination purpose of the subject, acquiring a scan parameter based on the specified scan region and examination purpose, and presenting the acquired scan parameter as part of a scan condition.

According to the present disclosure, it is possible to easily set a scan parameter even in a case where the number of protocols increases in accordance with the number of measurement modes in the photon counting CT.

### BRIEF DESCRIPTION OF THE DRAWINGS

FIG. 1 is a configuration diagram showing an example of a configuration of a radiation CT scan apparatus of an embodiment.

FIG. 2 is a configuration diagram showing an example of a configuration of a console of the embodiment.

FIG. 3 is a functional block diagram showing an example of a function of the console of the embodiment.

FIG. 4 is a flowchart showing an example of a flow of control processing of the embodiment.

### DETAILED DESCRIPTION

Hereinafter, an embodiment of the present invention will be described in detail with reference to the drawings. The present embodiment does not limit the present invention.

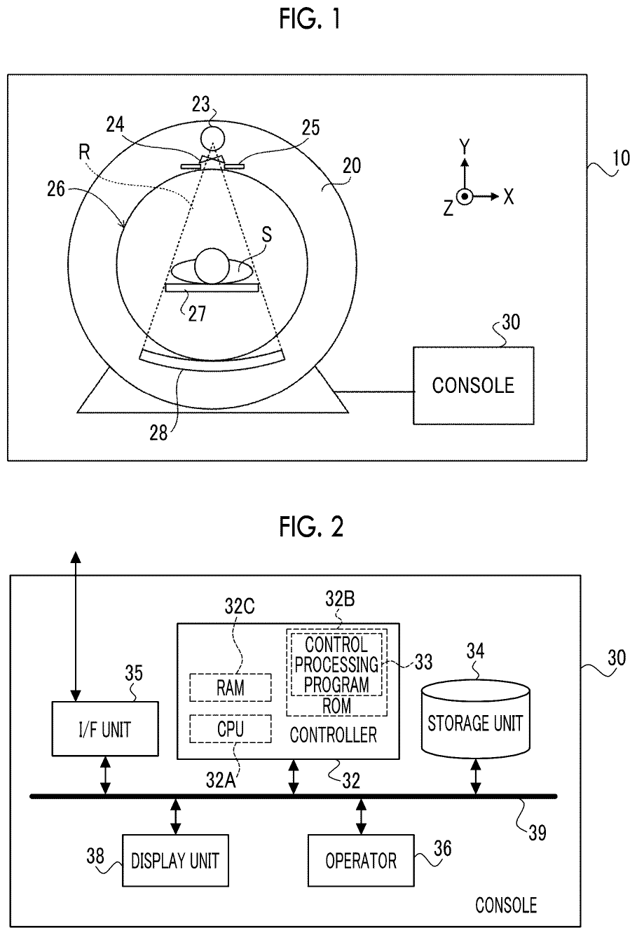

First, an example of a configuration of a radiation computed tomography (CT) scan apparatus of the present embodiment will be described. FIG. 1 is a configuration diagram showing an example of a configuration of a radiation CT scan apparatus 10 of the present embodiment. The radiation CT scan apparatus 10 of the present embodiment is an apparatus that performs CT scan for acquiring a plurality of pieces of projection data by using a photon counting type detector 28, which will be described in detail below. In the present embodiment, the CT in which the plurality of pieces of projection data are acquired by the photon counting type detector 28 in this way is referred to as “photon counting CT”.

As shown in FIG. 1, the radiation CT scan apparatus 10 of the present embodiment comprises a gantry 20, a bed 27, and a console 30.

The gantry 20 has an opening portion 26, and a subject S to be scanned is disposed in the opening portion 26 in a state of being placed on the bed 27. The gantry 20 and the bed 27 can be moved relatively in a direction penetrating the opening portion 26. The subject S of the present embodiment is an example of a subject of the present disclosure.

Inside the gantry 20, a radiation generation device 23 having a radiation tube (not shown), a bow tie filter 24, and a collimator 25, and the detector 28 are disposed in a state of facing each other across the subject S. Radiation R emitted from the radiation generation device 23 is formed into a beam shape suitable for a size of the subject S by the bow tie filter 24 and the collimator 25 and is emitted to the subject S. The detector 28 detects the radiation transmitted through the subject S to generate projection data in accordance with the dose of the detected radiation. The detector 28 of the present embodiment is a photon counting type detector in which a plurality of detection elements (not shown) that detect photon energy, which is the energy of the photon of the incident radiation, are arranged in an arc shape centered on the focus of a radiation tube of the radiation generation device 23. The detector 28, which is the photon counting type detector, outputs projection data corresponding to the photon energy.

The radiation generation device 23 and the detector 28 are rotated around the subject S by a rotation driving unit (not shown) of the gantry 20. The radiation irradiation from the radiation generation device 23 and the radiation detection of the detector 28 are repeated while both the radiation generation device 23 and the detector 28 are rotated, so that projection data is acquired at various projection angles. The plurality of pieces of projection data acquired by the detector 28 are reconstructed by an image reconstruction unit (not shown) of the console 30 and are output as an image.

The console 30 of the present embodiment performs various types of control related to a scan, generation of a medical image, and the like. The console 30 of the present embodiment is an example of a control device of the present disclosure. For example, the console 30 of the present embodiment is a server computer. As shown in FIG. 2, the console 30 comprises a controller 32, a storage unit 34, an interface (I/F) unit 35, an operator 36, and a display unit 38. The controller 32, the storage unit 34, the I/F unit 35, the operator 36, and the display unit 38 are connected to each other via a bus 39 such as a system bus or a control bus so as to be able to transmit and receive various types of information.

The controller 32 of the present embodiment controls the overall operation of the console 30. The controller 32 comprises a central processing unit (CPU) 32A, a read only memory (ROM) 32B, and a random access memory (RAM) 32C. Various programs including a control processing program 33, which will be described below, executed by the CPU 32A are stored in the ROM 32B in advance. The RAM 32C temporarily stores various types of data.

The storage unit 34 stores the projection data output from the detector 28, various other types of information, and the like. Specific examples of the storage unit 34 include a storage medium such as a hard disk drive (HDD), a solid state drive (SSD), and a flash memory.

The I/F unit 35 performs communication of various types of information with a rotation driving unit (not shown) of the gantry 20, the radiation generation device 23, and the detector 28 through wired communication or wireless communication. The console 30 of the present embodiment receives the projection data from the detector 28 via the I/F unit 35. The received projection data is stored in the storage unit 34.

The operator 36 is used by a user to input various types of information such as a scan condition for acquiring the projection data, an instruction related to the generation of the image such as an instruction of a parameter, and an instruction related to the display of the image. The operator 36 is not particularly limited, and examples thereof include various switches, buttons, a touch panel, a touch pen, a keyboard, and a mouse. The display unit 38 displays various types of information, a medical image, and the like. The operator 36 and the display unit 38 may be integrated into a touch panel display. In addition, for example, the operator 36 may receive a voice input from the user.

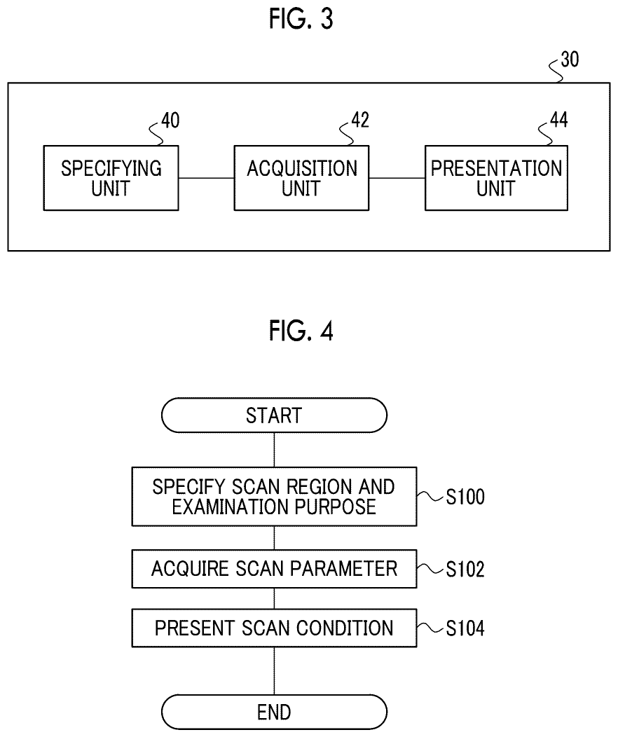

FIG. 3 shows a functional block diagram showing an example of a function of the console 30. The console 30 comprises a specifying unit 40, an acquisition unit 42, and a presentation unit 44. As an example, the console 30 of the present embodiment executes the control processing program 33, so that the CPU 32A of the controller 32 functions as the specifying unit 40, the acquisition unit 42, and the presentation unit 44.

The specifying unit 40 specifies a scan region and an examination purpose of the subject S. The specifying unit 40 outputs the specified scan region and examination purpose of the subject S to the acquisition unit 42. A method by which the specifying unit 40 specifies the scan region and the examination purpose of the subject S is not limited. For example, the specifying unit 40 may specify the scan region and the examination purpose of the subject S from the information input by the user through the operator 36. In addition, for example, the specifying unit 40 may specify the scan region and the examination purpose of the subject S from an imaging order, medical record information of the subject S, and the like.

The acquisition unit 42 acquires scan parameters based on the scan region and the examination purpose specified by the specifying unit 40. Here, an example of the method of acquiring the scan parameters in the acquisition unit 42 of the present embodiment will be described in detail as “Example 1”. The scan parameter of the present example includes a measurement mode and a focal size.

### Example 1

In the present example, a protocol of the energy-integrating radiation CT apparatus is used as a basis. Then, general scan parameters such as a scan type, a tube voltage, and a tube current are associated with the scan region, a measurement mode, which is a scan parameter unique to the photon counting CT, and a focal size closely related to the measurement mode from the viewpoint of the spatial resolution are grouped and assigned rules, and are associated with the examination purpose.

Table 1-1 shows an example of a feature of the measurement mode which is one of the scan parameters of the present example. The measurement mode is provided by the system by a combination of the spatial resolution in the X-Y plane, a minimum slice thickness (slice thickness: Z direction in FIG. 1) of the reconstructed tomographic image, and the energy information (specifically, the energy resolution), and corresponds to the determination of the contents of the projection data, that is, Raw Data, which is transmitted from the detector 28 to the console 30 in a case of scanning the subject S and that is used for the reconstruction of the image.

In the present example, the combination of the spatial resolution in the X-Y plane, the minimum slice thickness, and the energy information is set to fit within a certain data size from the relationship between the data transmission amount and the storage capacity. A measurement mode A is a mode in which both the spatial resolution in the X-Y plane and the minimum slice thickness are standard, but the amount of energy information is large and calibration is performed using a reference substance suitable for the measurement of a quantitative value. A measurement mode B is characterized in that the spatial resolution in the X-Y plane is the same as that in the measurement mode A, but a slice thickness thinner than that in the measurement mode A can be set. In the measurement mode B, the energy information is less than that in the measurement mode A due to the relationship of the amount of data. Measurement modes C and D are characterized in that data having a higher spatial resolution in the X-Y plane than the measurement mode A can be obtained by setting an appropriate focal size. The measurement mode C can be used with the same minimum slice thickness as the measurement mode A, and also allows the use of the energy information. On the other hand, in the measurement mode D, it is possible to set a slice thickness thinner than that in the measurement mode A, but there is no energy information. Measurement mode E is a mode in which the spatial resolution in the X-Y plane can be set higher and the minimum slice thickness can be set thinner than in measurement modes C and D. The measurement mode E is the same as the measurement mode D in that there is no energy information, but, due to higher spatial resolution in the X-Y plane and thinner minimum slice thickness, the amount of data increases, which imposes a limitation on the coverage over which data can be acquired at one time.

TABLE 1-1

| Scan parameter | Feature | | |
|---|---|---|---|
| Measurement | Spatial resolution | Minimum slice | Energy |
| mode | in X-Y plane | thickness | information |
| A | Standard resolution | Standard | Large |
| B | | Thin | Medium |
| C | Equal to or less than | Standard | Medium |
| D | high resolution | Thin | None |
| E | Equal to or less than | Ultra-thin | None |
| | ultra-high resolution | | |

In addition, Table 1-2 shows an example of a feature of the focal size, which is one of the scan parameters of the present example. The focal size is a parameter that affects the spatial resolution in the X-Y plane, and there is a focal size suitable for the spatial resolution in each measurement mode. For example, in the measurement mode A which is standard spatial resolution, even in a case where the focal size is set to “small”, spatial resolution equal to or higher than spatial resolution in a case where the focal size is set to “medium” cannot be obtained. In addition, as the focal size is smaller, the tube current that can be applied is limited. Therefore, in a case of the measurement mode A, there is no advantage in setting the focal size to “small” or “extremely small”. In addition, in the measurement mode E having ultra-high resolution, in a case where the focal size is set to “medium”, the spatial resolution equal to or higher than the spatial resolution in the measurement mode D cannot be obtained. Therefore, the rule is applied so that the focal size suitable for each measurement mode is set. Table 1-3 shows an example of a rule of an optimum focal size for each measurement mode. In a case where the focal size of the scan parameter is set to “optimum”, an optimum focal size (see Table 1-3) is set in accordance with the measurement mode. The optimum focal size is a focal size in which higher spatial resolution can be expected than in a case where other focal sizes are set.

TABLE 1-2

| Scan parameter | |
|---|---|
| Focal size | Feature |
| Large | Optimum for measurement modes A and B |
| Medium | Maintain difference of spatial resolution in X-Y |
| | plane in measurement modes A, B, C, and D |
| Small | Optimum for measurement modes C and D |
| | Maintain difference of spatial resolution in X-Y |
| | plane in measurement modes C, D, and E |
| Extremely small | Optimum for measurement mode E |

TABLE 1-3

| Scan parameter | | |
|---|---|---|
| Focal size | Rule | Set value |
| Optimum rule | Measurement mode A | Large |
| | Measurement mode B | Large |
| | Measurement mode C | Small |
| | Measurement mode D | Small |
| | Measurement mode E | Extremely |
| | | small |

The measurement modes and the focal size shown in Tables 1-1 to 1-3 are associated with the examination purpose. Table 1-4 shows an example of a correspondence relationship between the examination purpose and the measurement mode and the focal size.

TABLE 1-4

| | | | Scan parameter | |
|---|---|---|---|---|
| Examination | Identification | Priority | Measure- | Focal |
| purpose | information | level | ment mode | size |
| Quantitative | — | 1 | A | Optimum |
| value | | | | |
| Routine | Normal | 1 | B | Optimum |
| | Detailed | 2 | C | Medium |
| | examination | | | |
| | (energy) | | | |
| | High definition | 3 | D | Medium |
| Detailed | — | 1 | C | Medium |
| examination | | | | |
| Fracture (large | — | 1 | C | Medium |
| part) | | | | |
| Blood vessel | Normal | 1 | C | Medium |
| (Ca processing) | Large physique | 2 | B | Optimum |
| Stent lumen | — | 1 | D | Optimum |
| Blood vessel | — | 1 | D | Optimum |
| Fracture (small | — | 1 | E | Optimum |
| part) | | | | |

The examination purpose can be set to any word, and the examination purpose set here is selected in a case of reading out the protocol. As in a case where the examination purpose is “routine”, it is also possible to set a combination of a plurality of measurement modes and focal sizes by assigning a priority (priority level) to one examination purpose. In a case of reading out the protocol, in general, the protocol having the priority level of “1” is read out. In a case where a combination different from the combination in which the priority level is set to “1” is used, for example, the combination may be changed to another combination after confirming the use application described in the identification information such as “normal”, “examination (energy)”, and “high definition” without changing the examination purpose in a case of planning the scan.

For example, in a case where the examination purpose is the measurement of a “quantitative value”, a measurement mode A in which the energy information is large and calibration is performed with a reference substance suitable for measuring the quantitative value, and a focal size in which the focal size optimum for the measurement mode A is “large” are suitable.

In addition, in a case where the examination purpose is a general “routine” examination to check for the presence or absence of an abnormality, a measurement mode B in which the standard spatial resolution is sufficient, images can be reconstructed with thin slice thickness for multi planar reconstruction (MPR) or three dimensions (3D), and a virtual monochromatic radiation image that is capable of improving contrast improvement or reducing metal artifacts can be obtained as necessary, and the focal size, which is optimum for the measurement mode B, that is, “large” are suitable. In a case where the doctor gives priority to detailed examination even in the routine examination, the measurement mode C in which the spatial resolution is high, rather than obtaining the thin slice thickness, and the energy information can be used and the focal size “medium” are suitable. In addition, in a case where an image having a thin slice thickness and high resolution is desired even though the energy information is not available, the measurement mode D and the focal size “medium” are suitable.

In addition, in a case where the examination purpose is to suppress calcium (Ca) of a blood vessel (coronary artery) of the heart or to perform calcium scoring, which is “blood vessel (Ca processing)”, the calcium can be specified by the energy information, and the measurement mode C with high spatial resolution and the focal size “medium” are suitable. In a case where the physique of the subject S is large, it is necessary to set the focal size to “large” in order to increase the dose (tube current) in a case of the measurement mode C, and thus the spatial resolution decreases. Therefore, the significance of selecting the measurement mode C is reduced. Therefore, in a case where the physique of the subject S is large, the measurement mode B in which a thin slice thickness is obtained and the focal size which is the optimum for the measurement mode B, that is, “large” are suitable.

In addition, in a case where the examination purpose is the “stent lumen” for evaluating the stent lumen, the measurement mode D in which the spatial resolution is high and an image with a thin slice thickness can be reconstructed for MPR or 3D, and the focal size “small” are suitable. In addition, the same applies to a case where the examination purpose is “blood vessel” for observing the running state of the blood vessel.

In addition, since acute fractures can be diagnosed by observing the presence or absence of bone marrow edema using calcium-suppressed water density images, in a case where the examination purpose is “fracture (large part)”, energy information can be used and measurement mode C, which has high spatial resolution, and a focal size “medium” are suitable. On the other hand, in a case where the examination purpose is “fracture (small part)” in which a small part such as a hand is to be observed more precisely, the measurement mode E in which an ultra-high resolution image is obtained and the focal size in which the focal size suitable for the measurement mode E is “extremely small” are suitable.

In addition, the optimum focal size of the measurement modes C and D in the examination purpose of “routine” for “detailed examination (energy)” and “high definition”, and “detailed examination”, “fracture (large part)”, and “blood vessel (Ca processing)” for “normal” is “small”. While the above, in the examples shown in Table 1-4, in consideration of the dose (tube current) related to the signal/noise (S/N), the “medium” which is the focal size which has higher spatial resolution than the measurement modes A and B in which the spatial resolution is slightly decreased is expected.

In addition to the scan for obtaining the image for diagnosis, complementary scan types such as a scanogram for planning the scan and a monitoring scan for monitoring the degree of staining of the contrast agent is combined and registered in the protocol. The measurement mode and the focal size of these scan types are set separately as shown in Table 1-5.

TABLE 1-5

| Scan type | Measurement mode | Focal size |
|---|---|---|
| Scanogram | Same as diagnostic | Same as diagnostic |
| | scan type | scan type |
| Monitoring positioning | B | Optimum |
| Monitoring | B | Optimum |

Among the scan types, the “scanogram” is set to the same measurement mode and focal size as the scan type for diagnosis. In addition, for “monitoring positioning” and “monitoring”, the measurement mode B is set and the focal size, which is the optimum focal size for the measurement mode B, is set to “large”.

In the present example, the parameter of the reconstructed image (tomographic image) used for diagnosis is associated with the scan region, and in a case where settable scan parameters, such as the type of reconstructable image and spatial resolution, are changed in accordance with an examination purpose, that is, a measurement mode and a focal size, adjustment is performed to maintain, as much as possible, the type of image and parameters set in a protocol. For example, although the calcium-suppressed image requiring the energy information is set in the protocol associated with the scan region, in a case where the examination purpose is selected as “routine” and the priority level is changed to “high definition” of “3” (see Table 1-4), the measurement mode becomes the measurement mode D in which the energy information is not present, and the calcium-suppressed image cannot be obtained. However, in a case where the spatial resolution of the CT image is set to “highest”, an image with the high resolution of the measurement mode D is obtained instead of the standard resolution of the measurement mode B.

As described above, in the present example, since the scan parameters are associated with the scan region and the examination purpose, the console 30 can obtain information indicating a correspondence relationship. The acquisition unit 42 acquires the scan parameters corresponding to the scan region and the examination purpose of the subject S, based on the information indicating the correspondence relationship. The acquisition unit 42 outputs the acquired scan parameters to the presentation unit 44.

The presentation unit 44 presents the scan parameters acquired by the acquisition unit 42 as a scan condition. A presentation method by which the presentation unit 44 presents the acquired scan parameter as the scan condition, and a presentation destination are not limited. For example, the presentation unit 44 may present, on a device mounted on the gantry 20, for example, a touch pad or the like. In addition, scan parameters related to the reconstruction of the tomographic image, that is, the generation of the image may be presented to an image reconstruction unit (not shown) in the console 30. In addition, for example, the presentation unit 44 may present the scan conditions to the user by displaying the scan conditions on the display unit 38 of the console 30. The presentation destination at which the presentation unit 44 presents the scan condition may be one place or a plurality of places. In addition, for example, the presented scan condition may include an item other than the scan parameters. The presentation unit 44 may present, together with other information, the scan parameter acquired by the acquisition unit 42 by including the scan parameter in the scan condition.

Next, an action of the console 30 of the present embodiment will be described.

In the console 30 of the present embodiment, the CPU 32A of the controller 32 executes the control processing as an example shown in FIG. 4 by executing the control processing program 33 stored in the ROM 32B. FIG. 4 is a flowchart showing an example of a flow of the control processing in the console 30 of the present embodiment.

First, in Step S100 of FIG. 4, the specifying unit 40 specifies the scan region and the examination purpose of the subject S as described above.

In next Step S102, the acquisition unit 42 acquires the scan parameter, based on the scan region and the examination purpose of the subject S specified in Step S100 as described above.

In the next Step S104, the presentation unit 44 presents the scan parameter acquired in Step S102 as the scan condition, as described above. In a case where the processing of Step S104 ends, the control processing shown in FIG. 4 ends.

The present disclosed technology is not limited to the above-described embodiments, and can be adapted to various modification examples. In the following, examples that is a modification example of Example 1 will be described.

### Example 2

In the present example, a method of forming a protocol by grouping and assigning rules to each scan parameter based on its role and meaning and associating the scan parameter with a scan region or an examination purpose will be described. Table 2-1 shows an example of a set of scan parameters (hereinafter, referred to as a parameter set) associated with the scan region. In the example shown in Table 2-1, for example, a size (width, length, and height) covering the scan region, the presence or absence of the influence of the movement of the subject S or the scan region, and the influence of the bone are set as information indicating the feature of the scan region. In addition, a tube voltage, a tube current, a scan time, and a compensating object size are summarized as a “dose group”.

TABLE 2-1

| Scan parameter | | Description |
|---|---|---|
| Feature | Size | Set size (width (X direction), length (Z direction), height (Y |
| | | direction)) of scan region to be imaged as basic information. |
| | Influence of | Set part affected by movement such as breathing and heartbeat |
| | movement | |
| | Influence of bone | Set scan region affected by bone such as beam hardening |
| Dose | Tube voltage | Set reference value and adjust based on physique and age |
| | Tube current | Determine dose based on scan region and physique in accordance |
| | | with scan time |
| | Scan time | Set scan region that moves due to breathing and heartbeat, or the |
| | | like, or scan region affected by such movement as fast scan time |
| | | and set high absorption body such as bone or scan region |
| | | surrounded by high absorption body, which does not move as slow |
| | | scan time to increase dose |
| | Compensating | Set compensating object for adjusting field of radiation |
| | object size | irradiation based on size of scan region |

In addition, Table 2-2 shows a setting example of feature information assuming that the scan region is the “head”.

TABLE 2-2

| Scan parameter | Set value |
|---|---|
| Size | Width: 250 mm |
| (width, length, height) | Length: 150 mm |
| | Height: 250 mm |
| Influence of movement | None |
| Influence of bone | Present |

Further, Table 2-3 shows an example of a settable value in each scan parameter belonging to the dose group described above. In addition, Table 2-4 shows a setting example of an optimum rule of the tube voltage, which is one of the scan parameters.

TABLE 2-3

| Scan parameter | Settable value | Remark |
|---|---|---|
| Tube voltage | {70, 100, 120, 140 kV, | |
| | optimum} | |
| Tube current | {10 to 1000 mA, AEC} | AEC: Auto |
| | | Exposure Control |
| Scan time | {0.35, 0.4, 0.5, 1.0, 2.0 s, | |
| | automatic} | |
| Compensating | {Standard, small, automatic} | |
| object | | |

TABLE 2-4

| Scan parameter | | |
|---|---|---|
| Tube voltage | Rule | Set value |
| Optimum rule | Reference value | 120 kV |
| | Weight of 90 kg or more | 140 kV |
| | 10 years old or younger | 70 kV |

The tube voltage is set to 120 kV as a reference value from the viewpoint of a window level (WL) and a window width (WW) during interpretation. In a case where the physique of the subject S is large, 140 kV higher than the reference value is recommended to reduce the beam hardening artifact. In addition, for children, it is preferable that a value lower than the reference value, and 70 kV in which contrast can be obtained even in a case where the dose is reduced is recommended. In consideration of the recommended tube voltage in this way, an optimum tube voltage (kV) is automatically set from the weight and age information of the subject S.

The tube current, which is one of the scan parameters, is set to an optimum tube current in consideration of the setting of a standard deviation (SD), which is a standard deviation of a CT value, by auto exposure control (AEC) using the scanogram.

Table 2-5 shows a setting example of an automatic setting rule of the scan time, which is one of the scan parameters.

TABLE 2-5

| Scan parameter | | | Influence of bone | |
|---|---|---|---|---|
| Scan time | Rule | | None | Present |
| Automatic setting | Influence of | None | 0.5 s | 1.0 s |
| rule | movement | Present | 0.5 s | 0.5 s |

The scan time is set to 0.5 s, which is a fast scan time, for a scan region that moves due to breathing, a heartbeat, or the like or a scan region that is affected by the breathing, the heartbeat, or the like, and is set to 1.0 s, which is a slow scan time, for a high absorption body such as a bone or a scan region surrounded by the high absorption body, which does not move, in order to increase the dose. In a case where the scan region is the heart, it is set based on the heart rate.

Table 2-6 shows a setting example of an automatic setting rule of the compensating object size, which is one of the scan parameters.

TABLE 2-6

| Scan parameter | | |
|---|---|---|
| Compensating | | |
| object size | Rule | Set value |
| Automatic | Scan region size (width): FOV 200 mm or | Small |
| setting rule | less | |
| | Scan region size (width): FOV 201 mm or | Standard |
| | more | |

The compensating object size is determined based on the size (width) of the scan region.

The parameters of the dose group described above are set for each combination of the scan type, the measurement mode, and the focal size.

Table 2-7 shows a specific setting example of the dose group assumed to be associated with the scan region in a case where the scan region is “head”.

TABLE 2-7

| | | Measurement | | Tube | Tube | | | Compensating |
|---|---|---|---|---|---|---|---|---|
| Dose# | Scan type | mode | Focal size | voltage | current | | Scan time | object |
| DS-0001 | Scanogram | Common | Common | 120 kV | 50 | mA | — | Standard |
| | Conventional | A, B, C, D | Large, | Optimum | AEC | | Automatic | Automatic |
| | Helical | | medium | | | | | |
| | Dynamic | | | | | | | |
| | Monitoring | A | Large | 120 kV | 100 | mA | 1.0 s | Standard |

In a case where the scan type is a scanogram, the tube voltage, the tube current, and the compensating object are fixed values in any combination of the measurement modes and the focal sizes. In addition, in a case where the scan type is the conventional scan, the helical scan, and the dynamic scan, the measurement mode is any of A, B, C, and D, and in a case where the focal size is “large” or “medium”, the tube voltage is set to “optimum”, the tube current is set to “AEC”, and the scan time and the compensating object size are set to “automatic”. In a case where the scan type is the monitoring scan, the measurement mode is set to A, and the focal size is set to “large”. In a case where a combination that is not defined here is attempted to be set in the protocol, the user is prompted to either register the combination in the dose group or to set the combination as an individual parameter.

Table 2-8 shows a parameter set associated with an examination purpose defined for each scan region.

TABLE 2-8

| Parameter set | Description |
|---|---|
| Scan sequence | Flow of scan to be executed is determined depending on |
| frame | purpose of examination for each region. Measurement |
| | mode and focal size belong to this category. |
| Image set | Necessary diagnostic image is determined according to |
| | examination purpose. Settable value varies depending |
| | on measurement mode. |

The scan sequence frame is a parameter set related to a scan flow determined based on an examination purpose for each scan region. An image set is a parameter set related to a diagnostic image to be obtained by scanning. Since the parameters belonging to the image set are also parameters for reconstructing the projection data that is obtained by the scan, the parameters are also treated as the scan parameters.

Table 2-9 shows the settable values of the parameters belonging to the scan sequence frame described above.

TABLE 2-9

| Scan parameter | Settable value | Remark |
|---|---|---|
| Scan type | {Scanogram, conventional, helical, monitoring, | Scan method |
| | ECG-conventional, ECG-helical, . . . } | |
| Measurement | {A, B, C, D, E} | |
| mode | | |
| Focal size | {Large, medium, small, extremely small, optimum} | |
| Start mode | {Manual, Auto} | Whether start button operation is |
| | | required at starting |
| Link mode | {None, free, continuous, repetition, return} | Positional relationship of sequence |
| Scan direction | {Top to bottom, bottom to top, fixed} | Scan direction based on subject's body |
| Scan length | {Numerical value {minimum to maximum scan | Converted to number of scans and pitch |
| | length}, auto} | parameters based on scan length |
| Contrast | {OFF, ON} | Whether contrast agent is injected |
| Delay | {None, minimum, + value between sequences, | Time from end of previous sequence to |
| | accumulated + value} | start of main sequence |

Table 2-10 shows a setting example of the automatic setting rule for the scan length of the scanogram.

TABLE 2-10

| Scan parameter | | | Set |
|---|---|---|---|
| Scan length | Rule | | value |
| Automatic | Coronal | Scan region size: width ≥ length | Width |
| setting rule | | Scan region size: width < length | Length |
| | Sagittal | Scan region size: height ≥ length | Height |
| | | Scan region size: height < length | Length |

In the scanogram, the cross section can be imaged in a coronal and sagittal manner, and the scan length is set to a basic set value of the width or the height of the scan region in relation to the pixel size used for scanning. Then, in a case where a length (a length of the subject S in a body axis direction) is greater than a width or a height of the scan region, the scan length is adjusted to the length direction.

The parameter is for designating how to control the combination of each scan method, and a measurement mode unique to the photon counting CT and a focal size closely related to the measurement mode are also included in the scan sequence frame.

Table 2-11 shows a specific setting example of the scan sequence frame for each examination purpose in a case where the scan region is the “head”. The “purpose” in Table 2-11 indicates an examination purpose assumed for association. The actual association is shown in Table 2-15 described later.

TABLE 2-11
Sequence

| frame# | | Measurement | | Start | Link | Scan | Scan | | | |
|---|---|---|---|---|---|---|---|---|---|---|
| “purpose” | Scan type | mode | Focal size | mode | mode | direction | length | | Contrast | Delay |
| SF-0001 | Scanogram | Same as scan | Same as | Manual | None | Bottom | Automatic | | OFF | Minimum |
| | | | scan | | | to top | | | | |
| | Conventional | Setting for | Setting for | Manual | None | Bottom | 120 | mm | OFF | Minimum |
| | | each purpose | each purpose | | | to top | | | | |
| Sub-0001 | | B | Optimum | | | | | | | |
| “routine” | | | | | | | | | | |
| Sub-0002 | | C | Medium | | | | | | | |
| “detailed | | | | | | | | | | |
| examination” | | | | | | | | | | |
| SF-0002 | Scanogram | C | Optimum | Manual | None | Bottom | Automatic | | OFF | Minimum |
| “cerebral blood | | | | | | to top | | | | |
| vessel” | Dynamic | B | Optimum | Manual | None | Fixed | 40 | mm | OFF | Minimum |
| | Monitoring | B | Optimum | Manual | None | Fixed | 40 | mm | ON | Sequence |
| | | | | | | | | | | start 8 s |
| | Helical | C | Medium | Auto | Free | Bottom | 80 | mm | ON | Minimum |
| | | | | | | to top | | | | |
| SF-0003 | Scanogram | B | Optimum | Manual | None | Bottom | Automatic | | OFF | Minimum |
| “blood flow” | | | | | | to top | | | | |
| | Dynamic | B | Optimum | Manual | None | Fixed | 40 | mm | ON | Minimum |

“SF-0001” of the sequence frame in Table 2-11 corresponds to a general routine, and the scan type includes two types of “scanogram” and “conventional”. The measurement mode and the focal size of the scanogram are set to be the same as those of the diagnostic scan. In addition, a measurement mode and a focal size of the conventional scan type are designated as “setting for each purpose”, which is configured to be set for each examination purpose. In a case where the “setting for each purpose” is designated, it is possible to set a plurality of “examination purposes” separately, and it is possible to set in more detail by “Sub-****” of the sequence frame for each examination purpose. In “Sub-0001” in the setting example shown in Table 2-11, the measurement mode B is set and the focal size is set to “optimum”, that is, “large” assuming the association with “routine” for the examination purpose. In addition, in “Sub-0002”, the measurement mode C is set and the focal size “medium” is set assuming the association with the “detailed examination” for the examination purpose. In addition, other parameters (empty frames in Table 2-11) in “Sub-0001” and “Sub-0002” inherit the setting of “SF-0001”.

Although not shown in the setting example described above, for example, by adding “Sub-0003” for association with the examination purpose “detailed examination (contrast)” and setting the contrast to “ON”, it is also possible to overwrite the parameters inherited from “SF-0001”. It is also possible to use a method in which “SF-0001” that serves as a base of the setting for each purpose is associated with the scan region instead of the examination purpose, common parameters are inherited from the scan region, and parameters that need to be changed in accordance with the examination purpose are set for each purpose.

In addition, in “SF-0001”, “Sub-0001”: “routine” and “Sub-0002”: “detailed examination”, and “SF-0002”: “cerebral blood vessel” and “SF-0003”: “blood flow”, the scan length of the scanogram is set to “automatic”, and the scan region information, that is, the setting is determined based on the “size (width, length, height)” information in the set of scan parameters associated with the scan region shown in Table 2-1.

Here, a case where it is desired to adjust a part or all of the parameters (dose information group) associated with the scan region according to the examination purpose is conceivable. In this case, the parameters can be set in the scan sequence frame, and the setting is prioritized. For example, in a case where a tube voltage is set in the scan sequence frame, the setting is prioritized.

Table 2-12 shows an example of a settable value of the scan parameter belonging to the image set described above.

TABLE 2-12

| Scan | | |
|---|---|---|
| parameter | Settable value | Remark |
| Image type | {CT image, virtual monochromatic | Selection varies |
| | X-ray image, calcium-suppressed | depending on |
| | image, iodine-suppressed image, | measurement mode |
| | iodine map, . . . } | |
| FOV | {20 to 500 mm} | |
| Slice | {0.25 to 10 mm, minimum, | Selection varies |
| thickness | maximum} | depending on |
| | | measurement mode |
| Matrix size | {512, 1024, 2048, automatic} | |
| Spatial | {Standard, high, highest} | |
| resolution | | |
| Reconstruction | {Select depending on use} | |
| filter | | |
| Correction | Numerical value {minimum to | |
| | maximum scan length} | |
| Window | {Window value (window width, | |
| (WW/WL) | window level)} | |
| Automatic | {None, 3D, MPR, . . . } | |
| analysis | | |
| Transfer | {None, PACS, WS, . . . } | |
| destination | | |

The scan parameters belonging to the image set are parameters for designating what type of diagnostic image (tomographic image) is to be created, and also include designation of processing (automatic analysis such as 3D, MPR, and an image transfer destination) for the generated image.

Table 2-13 shows a specific setting example of the image set for each examination purpose in a case where the scan region is the “head”. In addition, Table 2-14 shows a setting example of a table for automatic setting of the matrix size, which is the size of the entire set of pixels that constitutes the tomographic image.

TABLE 2-13

| Image set# | Image | | Slice | | Matrix | Spatial | Reconstruction | | Window | Automatic | Transfer |
|---|---|---|---|---|---|---|---|---|---|---|---|
| “purpose” | information | FOV | thickness | | size | resolution | filter | Correction | (WW/WL) | analysis | destination |
| IM-0001 | CT image | 240 mm | 5 | mm | Automatic | Highest | H01 | None | 80/40 | None | PACS |
| “routine” | | | | | | | | | | | |
| “detailed | | | | | | | | | | | |
| examination” | | | | | | | | | | | |
| “cerebral | | | | | | | | | | | |
| blood vessel” | | | | | | | | | | | |
| IM-0002 | Virtual | 240 mm | 10 | mm | 512 | Standard | H02 | None | 150/40 | None | WS |

| | |
|---|---|
| “blood flow” | monochromatic |
| | X-ray image (70 |
| | keV) |

TABLE 2-14

| Standard resolution | | High resolution | | Ultra-high resolution | |
|---|---|---|---|---|---|
| | Matrix | | Matrix | | Matrix |
| FOV | size | FOV | size | FOV | size |
| 250 or less | 512 | 150 or less | 512 | 50 or less | 512 |
| 251 or more | 1024 | 151 or more | 1024 | 51 or more | 1024 |
| 400 or more | 2048 | 350 or more | 2048 | 150 or more | 2048 |

In the image set “IM-0001” in Table 2-13, parameters for reconstructing the CT image are set, the matrix size is set to “automatic”, and the spatial resolution is set to “highest”. In a case of the measurement mode A, a CT image of “standard resolution” which is the highest resolution in the measurement mode A is reconstructed with a matrix size (specifically, 512×512) of “512”, which is the setting in a case where the field of view (FOV) is “240” with reference to the matrix size automatic setting table of Table 2-14. In addition, in a case of the measurement mode C, similarly, the CT image of “high resolution” which is the highest resolution in the measurement mode C is reconstructed with a matrix size (specifically, 1024×1024) of “1024”, which is the setting in a case where the FOV is “240”.

On the other hand, in the image set “IM-0002” in Table 2-13, the virtual monochromatic radiation image (70 keV) of the “standard resolution” is reconstructed with a matrix size of “512” in any measurement mode as long as the measurement mode is the energy-available measurement modes A, B, and C.

Table 2-15 shows the content of the protocolization of the parameter set that is grouped into the dose group, the scan sequence frame, and the image set in association with the scan region and the examination purpose.

TABLE 2-15

| | | | | Scan | |
|---|---|---|---|---|---|
| Scan | | | Examination | sequence | Image |
| region | Size | Dose | purpose | frame | set |
| Head | Width: 250 mm | DS-0001 | Routine | SF-0001 | IM-0001 |
| | Length: 150 mm | | | Sub-0001 | |
| | Height: 250 mm | | Detailed | SF-0001 | IM-0001 |
| | | | examination | Sub-0002 | |
| | | | Cerebral | SF-0002 | IM-0001 |
| | | | blood vessel | | |
| | | | Blood flow | SF-0003 | IM-0002 |

As described above, even in the present example, since the parameter set of the scan parameter is associated with the scan region and the examination purpose, the console 30 can obtain information indicating the correspondence relationship. The acquisition unit 42 of the console 30 acquires the scan parameters corresponding to the scan region and the examination purpose of the subject S, based on the information indicating the correspondence relationship. The acquisition unit 42 outputs the acquired scan parameter to the presentation unit 44.

### Example 3

In the present example, an example of a flow of the examination using the protocol described in Example 1 will be described.

First, subject information (name, gender, age, and the like), a scan posture (head-first/feet-first and supine/prone/right decubitus/left decubitus), scan regiona scan region, and an examination purpose are set. The scan posture can also be set by obtaining information from a 3D camera. Table 3-1 shows a setting example of the subject information, the scan posture, the scan region, and the examination purpose.

TABLE 3-1

| | | | Scan | Scan | Examination |
|---|---|---|---|---|---|
| Name | Gender | Age | posture | region | purpose |
| AA BB | Female | 60 | Head-first/ | Heart | Blood vessel (Ca |
| | | | supine | | processing) |

The specifying unit 40 specifies “heart” as the scan region and “blood vessel (Ca processing)” as the examination purpose, and thus the acquisition unit 42 acquires protocols shown in Tables 3-2 to 3-4 as the scan parameters. Then, the presentation unit 44 presents the protocol. As an example, in the present example, the protocol is displayed on the display unit 38. Tables 3-2 to 3-4 show examples of protocols associated with a combination of the scan region “heart” and the examination purpose “blood vessel (Ca processing)”.

TABLE 3-2

| Scan | | Tube | Tube | | Scan | | | |
|---|---|---|---|---|---|---|---|---|
| region | Scan type | voltage | current | Scan time | length | | Contrast | : |
| Heart | Scanogram | 120 kV | 50 mA | — | 350 | mm | OFF | : |
| | Monitoring | 120 kV | 50 mA | 0.35 s/rot | 40 | mm | OFF | : |
| | positioning | | | | | | | |
| | Monitoring | 120 kV | 50 mA | 0.35 s/rot | 40 | mm | ON | : |
| | ECG-helical | 120 kV | AEC | 0.35 s/rot | 200 | mm | ON | : |

(430 mA)

TABLE 3-3

| | | | Parameter | |
|---|---|---|---|---|
| | Identification | Priority | Measurement | Focal |
| Purpose | information | level | mode | size |
| Blood vessel (Ca | Normal | 1 | C | Medium |
| processing | Large physique | 2 | B | Optimum |

TABLE 3-4

| Scan | Measurement | Focal |
|---|---|---|
| type | mode | size |
| Scanogram | Same as diagnostic | Same as diagnostic |
| | scan type | scan type |
| Monitoring positioning | B | Optimum |
| Monitoring | B | Optimum |

In the example of Table 3-4, the measurement mode of the scanogram is set to “same as diagnostic scan type”, and the focal size is also set to “same as diagnostic scan type”. Therefore, the measurement mode C, which is the measurement mode in which the scan type is set to “ECG (electrocardiogram)-helical”, is set and the focal size is set to “medium”.

After a start position of the scanogram is set, the scanogram of the subject S is acquired.

After the scan of the scanogram, a monitoring positioning scan is performed to determine a scan position for monitoring the contrast agent. As the measurement mode and the focal size are set in advance, the measurement mode is set to the measurement mode B, and the focal size is set to “optimum”, that is, the focal size is set to “large”.

After the scan of the monitoring positioning scan, the contrast setting is performed. Here, although the plan is to perform the monitoring scan with the measurement mode B and the focal size set to “optimum”, that is, “large”, and to perform the ECG-helical scan with the measurement mode C and the focal size set to “medium”, in a case where a current value reaches an upper limit value in the focal size set in the automatic tube current control (AEC) set for the ECG-helical scan, the scan is performed by switching the scan parameters (scan conditions). For example, the user clicks a right button of a mouse, which is an example of the operator 36, on the examination purpose “blood vessel (Ca processing)” displayed on the display unit 38, and selects “large physique” having a priority level of “2” from a displayed menu. As a result, the measurement mode associated with “large physique” is switched to the measurement mode B and the focal size associated with “large physique” is switched to “optimum” as shown in Table 3-3. As a result, the scan can be performed by switching to the scan condition in which the focal size is “large” and the upper limit of the tube current is high.

After the ECG (electrocardiogram)-helical scan, a diagnosis image set in the protocol is registered as a queue job, and a reconstructed tomographic image is obtained as a diagnosis image.

As described above, according to the present example, the CT scan of the subject S is performed according to the scan condition presented by the console 30 of the present disclosure, and the diagnosis image can be obtained, so that the examination of the subject S can be performed.

### Example 4

In the present example, an example of a flow of the examination using the protocol described in Example 2 will be described.

First, subject information (name, gender, age, and the like), a scan posture (head-first/feet-first and supine/prone/right decubitus/left decubitus), a scan region, and an examination purpose are set. The scan posture can also be set by obtaining information from a 3D camera. Table 4-1 shows a setting example of the subject information, the scan posture, the scan region, and the examination purpose.

TABLE 4-1

| | | | | | | Scan | Examination |
|---|---|---|---|---|---|---|---|
| Name | Gender | Age | Height | Weight | Scan posture | region | purpose |
| CC DD | Male | 50 | 170.0 cm | 70.0 kg | Head-first/supine | Head | Routine |

The specifying unit 40 specifies “head” as the scan region and “routine” as the examination purpose, and thus the acquisition unit 42 acquires protocols shown in Tables 4-2 to 4-10 as the scan parameters. Then, the presentation unit 44 presents the protocol. As an example, in the present example, the protocol is displayed on the display unit 38. Tables 4-2 to 4-10 show examples of protocols associated with a combination of the scan region “head” and the examination purpose “routine”.

TABLE 4-2

| Scan | | Influence of | Influence of | | Examination | Scan sequence | |
|---|---|---|---|---|---|---|---|
| region | Size | movement | bone | Dose | purpose | frame | Image set |
| Head | Width: 250 mm | None | Present | DS-0001 | Routine | SF-0001 | IM-0001 |
| | Length: 150 mm | | | | | Sub-0001 | |
| | Height: 250 mm | | | | | | |

TABLE 4-3

| | | Measurement | | Tube | Tube | | Compensating |
|---|---|---|---|---|---|---|---|
| Dose# | Scan type | mode | Focal size | voltage | current | Scan time | object |
| DS-0001 | Scanogram | Common | Common | 120 kV | 50 mA | — | Standard |
| | Conventional | B | Large | Optimum | AEC | Automatic | Automatic |

TABLE 4-4

| Sequence frame# | | Measurement | | Start | Link | Scan | Scan | | |
|---|---|---|---|---|---|---|---|---|---|
| “purpose” | Scan type | mode | Focal size | mode | mode | direction | length | Contrast | Delay |
| SF-0001 | Scanogram | Same as scan | Same as scan | Manual | None | Bottom to | Automatic | OFF | Minimum |
| | | | | | | top | | | |
| | Conventional | Setting for | Setting for | Manual | None | Bottom to | 120 mm | OFF | Minimum |
| | | each purpose | each purpose | | | top | | | |
| Sub-0001 | | B | Optimum | | | | | | |
| “routine” | | | | | | | | | |

TABLE 4-5

| Scan parameter | | Set | |
|---|---|---|---|
| Tube voltage | Rule | value | |
| Optimum rule | Reference value | 120 | kV |
| | Weight of 90 kg | 140 | kV |

| or more | | |
|---|---|---|
| 10 years old or | 70 | kV |

younger

TABLE 4-6

| Scan | |
|---|---|
| parameter | Influence of bone |

| | | | |
|---|---|---|---|
| Scan time | Rule | None | Present |

| | | | | |
|---|---|---|---|---|
| Automatic | Influence of | None | 0.5 s | 1.0 s |
| setting rule | movement | Present | 0.5 s | 0.5 s |

TABLE 4-7

| Scan parameter | | |
|---|---|---|
| Compensating | | Set |
| object size | Rule | value |
| Automatic | Scan region size (width): | Small |
| setting rule | FOV 200 mm or less | |
| | Scan region size (width): | Standard |
| | FOV 201 mm or more | |

TABLE 4-8

| Scan parameter | | | Set |
|---|---|---|---|
| Scan length | Rule | | value |
| Automatic | Coronal | Scan region size: width ≥ length | Width |
| setting rule | | Scan region size: width < length | Length |
| | Sagittal | Scan region size: height ≥ length | Height |
| | | Scan region size: height < length | Length |

TABLE 4-9

| Image set# | Image | | Slice | Matrix | Spatial | Reconstruction | | Window | Automatic | Transfer |
|---|---|---|---|---|---|---|---|---|---|---|
| “purpose” | information | FOV | thickness | size | resolution | filter | Correction | (WW/WL) | analysis | destination |
| IM-0001 | CT image | 240 mm | 5 mm | Automatic | Highest | H01 | None | 80/40 | None | PACS |
| “routine” | | | | | | | | | | |

TABLE 4-10

| Spatial resolution | | High resolution | | Ultra-high resolution | |
|---|---|---|---|---|---|
| | Matrix | | Matrix | | Matrix |
| FOV | size | FOV | size | FOV | size |
| 250 or less | 512 | 150 or less | 512 | 50 or less | 512 |
| 251 or more | 1024 | 151 or more | 1024 | 51 or more | 1024 |
| 400 or more | 2048 | 350 or more | 2048 | 150 or more | 2048 |

The scan parameters of the scanogram for planning a scan position and a scan range are respectively determined based on a preset rule or a set value, such that the measurement mode is a measurement mode B same as the diagnostic scan (conventional scan), the focal size is “optimum”, that is, the focal size is “large”, the tube voltage is 120 kV, the tube current is 50 mA, and the scan length is 250 mm based on size information associated with the scan region.

The scan direction is a head-first setting, and the bed 27 is controlled in a direction away from the scanner so that scan is performed from a lower side (legs) to an upper side (head) of the body.

The start position of the scanogram is determined by adjusting the position of the bed 27 with reference to the light projector while checking the subject S. Alternatively, the scanogram start position may be automatically set by using camera information.

In a case where the scanogram start position is set, the scanogram is acquired. The scanogram is acquired in real time for the purpose of confirming that the intended scan has been performed and stopping the scan in a case where the scan has been performed up to the necessary region, so it is imaged with the amount of data obtained by down-sampling the spatial resolution in the measurement mode B.

After the scanogram scan, a virtual monochromatic radiation image is generated by detailing (increasing the definition) to the spatial resolution of the measurement mode B depending on the measurement mode of the scanogram or by using the energy information, and an image useful for a scan plan in which the contrast is improved or the artifacts are reduced is regenerated. Accordingly, it is possible to confirm additional information.

A scan condition based on information obtained from an image obtained by the scanogram imaging is adjusted. For example, the scan start position can be set, or the “examination purpose” can be changed from the confirmation result of the scanogram image generated in post-processing. Here, in a case where the scan start position is set on the scanogram and the scan is performed according to the displayed protocol, the scan is performed under the scan conditions of the conventional scan in the measurement mode B, the focal size “large”, the tube voltage 120 kV, the tube current value by AEC, the scan time 1.0 s, the compensating object “standard”, and the scan length of 120 mm from the lower side (legs) to the upper side (head) of the body.

The scanner system operates in accordance with the scan parameter by an operation of confirming the scan parameter (start of scan preparation) by the user. In a case where the scan preparation is completed, the scan is started by the scan start operation. During scanning, a preview image is displayed on the display unit 38 of the console 30, and the user can confirm whether scanning is performed at an intended position.

In a case in which the scan is completed, a CT image, with a preset parameter, that is, a FOV of 240 mm at the standard resolution, which is the highest resolution of measurement mode A, is reconstructed by a queue job with a matrix size of 512×512. In addition, the reconstructed CT image is transferred to a picture archiving and communication system (PACS), which is a set transfer destination.

As described above, according to the present example, the CT scan of the subject S is performed according to the scan condition presented by the console 30 of the present disclosure, and the CT image can be obtained, so that the examination of the subject S can be performed.

### Example 5

In the photon counting CT, calibration is required for each measurement mode in principle. Therefore, in a device having a large number of measurement modes, it is conceivable that the device is used in a state in which maintenance work takes time and calibration is performed only for some measurement modes. There are various types of calibration. For example, there is a calibration in which the size of the base substance is changed and the photon count values under various measurement conditions such as the tube voltage and the tube current are measured and the results are tabulated in order to specify the substance using the energy information. In addition, for example, there is calibration in which circular phantoms having different sizes are measured under various measurement conditions to determine correction parameters in order to satisfy uniformity of the CT values regardless of the size of the substance. As described above, since there are a plurality of types of calibration, in the present example, first, a correspondence relationship between a measurement mode and a system state (available calibration state table and detector element state table) is set as shown in Table 5-1. Further, for each of a plurality of types of calibration, information indicating whether the calibration has been performed or not is obtained for each calibration state table. As an example, Table 5-2 shows whether each of the calibration 1 to n has been performed or not in the calibration state table (A). In addition, Table 5-3 shows whether each of the calibration 1 to n has been performed or not in the calibration state table (B). In Tables 5-2 and 5-3, “Completed” indicates that calibration has been performed, “Not completed” indicates that calibration has not been performed, and “-” indicates that there is no need to perform the calibration.

Based on the correspondence relationship shown in Table 5-1, a measurement mode that cannot be used is determined from the calibration state table corresponding to the measurement mode used for scanning, and in a case where the measurement mode is in an unavailable state in a case of reading out the protocol, control is performed to use a measurement mode close to the unavailable measurement mode. For example, according to Tables 5-2 and 5-3, in a state in which the calibration 2 required for the measurement mode A is not performed, the control is performed such that the next measurement mode B is used instead of the measurement mode A. However, in a case where the reconstructed image that cannot be executed in the measurement mode B is set, the fact is presented to the user.

In addition, there is a case where a defect occurs in a pixel of a detector element of the detector 28. Therefore, the measurement mode may be controlled according to a state of the detector element. In the present example, as the detector element state table, information indicating whether or not each pixel of the detector element of the detector 28 is available, such as whether or not it is defective, is obtained in advance. As an example, Table 5-4 shows whether or not each pixel is available for each pixel indicated by row×channel (CH) in the detector element state table (D). In addition, Table 5-5 shows whether or not each pixel is available for each pixel indicated by row×channel (CH) in the detector element state table (E). In addition, “A” in Tables 5-4 and 5-5 indicates that the pixel is not defective and is available, “B” indicates that the pixel is available but may be deteriorated, and “C” indicates that the pixel is unavailable.

For example, the use of the measurement mode E of the ultra-high resolution is not allowed, but there may be no problem in the measurement mode D of high resolution in which pixels are bundled. Therefore, the detector element state is determined from the detector element state table, and the control is performed to use the measurement mode close to the examination purpose.

TABLE 5-1

| | |
|---|---|
| Scan parameter | System state |

| Measurement | | Detector |
|---|---|---|
| mode | Calibration | element |
| A | Calibration state table (A) | Detector element state table (A) |
| B | Calibration state table (B) | Detector element state table (B) |
| C | Calibration state table (C) | Detector element state table (C) |
| D | Calibration state table (D) | Detector element state table (D) |
| E | Calibration state table (E) | Detector element state table (E) |

TABLE 5-2

| Calibration | | | | |
|---|---|---|---|---|
| state table | Calibration 1 | Calibration 2 | . . . | Calibration n |
| (A) | Completed | Not completed | . . . | Completed |

TABLE 5-3

| Calibration | | | | |
|---|---|---|---|---|
| state table | Calibration 1 | Calibration 2 | . . . | Calibration n |
| (B) | Completed | — | . . . | Completed |

TABLE 5-4

| Detector element | | | | | | |
|---|---|---|---|---|---|---|
| state table | | | CH1 | CH2 | . . . | CHm |
| (D) | Energy 1 | Row 1 | A | A | A | A |
| | | Row 2 | A | A | A | A |
| | | . | A | A | A | A |
| | | . | | | | |
| | | . | | | | |

TABLE 5-5

| Detector | | | | | | | | |
|---|---|---|---|---|---|---|---|---|
| element | | | | | | | | |
| state table | | | CH1 | CH2 | . . . | CHm | . . . | CHn |
| (E) | Energy 1 | Row 1 | A | A | A | A | A | A |
| | | Row 2 | A | A | C | A | B | A |
| | | . | A | A | B | A | C | A |
| | | . | | | | | | |
| | | . | | | | | | |
| | | | A | A | A | A | C | A |

Further, in the radiation generation device 23, the available focal size may be limited due to the non-execution of calibration using the focal size, the adjustment deviation of the focal size itself, or the like. Therefore, a correspondence relationship between the focal size and the system state (the use availability state table of the radiation tube) as shown in Table 5-6 is set, and further, the use availability of the focal size is obtained in advance for each use availability state table (Table 5-7). In Table 5-7, “O” indicates that it is available for use, and “X” indicates that it is not available for use.

In a case where it is determined that an appropriate focal size cannot be used in using the measurement mode from the use availability state corresponding to each focal size as shown in Table 5-6, the control is performed to use a set of the measurement mode and the focal size close to the spatial resolution for the examination purpose. In a case where the use availability is “X” in the use availability state table (extremely small) as shown in Table 5-7, that is, the focal size (extremely small) is unavailable, and in a case where the measurement mode E is set and the focal size is set to the extremely small, the focal size closest to the available focal size having a use availability state of “O”, that is, “(small)” which is available, is selected, and the measurement mode D which is most suitable for that focal size is set.

TABLE 5-6

| Scan | |
|---|---|
| parameter | System state |
| Focal size | Radiation tube |
| Large | Use availability state table (large) |
| Medium | Use availability state table (medium) |
| Small | Use availability state table (small) |
| Extremely | Use availability state table |
| small | (extremely small) |

TABLE 5-7

| Use availability | Use |
|---|---|
| state table | availability |
| (Large) | ◯ |
| (Medium) | ◯ |
| (Small) | ◯ |
| (Extremely small) | X |

As described above, according to the console 30 of the present modification example, the system state for the CT scan can be specified, and the scan parameters can be acquired further based on the specified state.

As described above, the console 30 is a control device used for CT scanning in which a plurality of pieces of projection data of the subject S are captured by using a photon counting type radiation detector, and the specifying unit 40 specifies the scan region and the examination purpose of the subject S. In addition, the acquisition unit 42 acquires the scan parameters based on the specified scan region and examination purpose. Then, the presentation unit 44 presents the acquired scan parameters as the scan condition.

In the energy-integrating radiation CT apparatus in the related art, an examination protocol is registered in association with scan regiona scan region, and is read out according to the scan region to be examined, and necessary conditions, a scan length, and the like are adjusted for use. In the detector of the photon counting CT apparatus, since it is possible to bundle the basic element size and the number of energy bins, it is common to prepare a plurality of systems for a combination (measurement mode) of the spatial resolution (the number of channels) in the X-Y plane, the slice thickness and the number of rows in the Z direction, and the number of energy bins, and to allow a user to select the combination. Therefore, the photon counting CT is equivalent to being able to switch CT apparatuses having different features, and in simple terms, the number of examination protocols is multiplied by the number of measurement modes. Therefore, it is not practical to individually set the parameters for the protocol considered necessary for the examination as in the related art and to register the parameters in association with the scan region.

On the other hand, as shown in each of the above examples, in the console 30 of the present embodiment, the parameters are structured (grouped) and associated rules, and the parameters are divided into those determined by the “scan region” and those determined by the “examination purpose” and associated with each other, so that the management of the protocol is facilitated. For example, the examination protocol is determined based on the “scan region” which corresponds to the position (location) on the human body, and the “examination purpose” which is what is desired to be seen at that scan region (such as confirming the overall condition of the scan region, confirming the state of blood vessel narrowing, confirming the state of blood flow).

Therefore, according to the console 30 of the present embodiment, in the photon counting CT, even in a case where the number of protocols increases according to the number of measurement modes, it is possible to easily set the scan parameters.

In each of the above-described embodiments, the following various processors can be used as the hardware structure of processing units performing various processes such as the specifying unit 40, the acquisition unit 42, and the presentation unit 44. As described above, the various processors include a programmable logic device (PLD) as a processor of which the circuit configuration can be changed after manufacture, such as a field programmable gate array (FPGA), a dedicated electrical circuit as a processor having a dedicated circuit configuration for executing specific processing such as an application specific integrated circuit (ASIC), and the like, in addition to the CPU as a general-purpose processor that functions as various processing units by executing software (program).

One processing unit may be configured of one of the various processors, or may be configured of a combination of the same or different types of two or more processors (for example, a combination of a plurality of FPGAs or a combination of the CPU and the FPGA). Further, a plurality of processing units may be composed of one processor.

As an example in which a plurality of processing units are configured of one processor, first, as typified by a computer such as a client or a server, one processor is configured of a combination of one or more CPUs and software, and this processor functions as a plurality of processing units. Second, as typified by a system on chip (SoC) or the like, a processor that realizes the functions of the entire system including the plurality of processing units by using one integrated circuit (IC) chip is used. In this way, various processing units are formed using one or more of the above-mentioned various processors as hardware structures.

Furthermore, as the hardware structure of these various processors, more specifically, an electric circuit (circuitry) in which circuit elements such as semiconductor elements are combined can be used.

In addition, in the above-described embodiment, the aspect has been described in which the control processing program 33 is stored (installed) in the storage unit 34 of the console 30 in advance, but the present invention is not limited to this. The control processing program 33 may be recorded on a recording medium, such as a compact disc read only memory (CD-ROM), a digital versatile disc read only memory (DVD-ROM), or a universal serial bus (USB) memory, and then provided. In addition, the control processing program 33 may be downloaded from an external device through a network.

In addition, the configurations and operations of the radiation CT scan apparatus 10, the console 30, and the like described in each of the above-described embodiments are merely examples, and it is needless to say that they can be changed according to the situation without departing from the gist of the present invention. In addition, it is needless to say that the above-described embodiments may be combined as appropriate.

In addition, the present invention can also be applied to a program and a program product.

In regard to the embodiment described above, the following supplementary notes will be further disclosed.

### Supplementary Note 1

A control device used in a CT scan that acquired a plurality of pieces of projection data of a subject by a photon counting type radiation detector, the control device comprising at least one processor configured to:
specify a scan region and an examination purpose of the subject;
acquire an scan parameter based on the specified scan region and examination purpose; and
present the acquired scan parameter as part of a scan condition.

### Supplementary Note 2

The control device according to Supplementary Note 1,
wherein the scan parameter is associated with at least one of the scan region or the examination purpose based on a rule.

### Supplementary Note 3

The control device according to Supplementary Note 2,
wherein the scan parameter includes a plurality of types, and
the processor is configured to acquire a first scan parameter in which a parameter value is associated based on a first rule corresponding to the scan region and a second scan parameter in which a parameter value is associated based on a second rule corresponding to the examination purpose.

### Supplementary Note 4

The control apparatus according to Supplementary Note 3,
wherein the processor is configured to present the second scan parameter as part of the scan condition, in a case where a type of the first scan parameter and a type of the second scan parameter are the same.

### Supplementary Note 5

The control device according to Supplementary Note 3,
wherein the second scan parameter includes at least one of a measurement mode or a focal size.

### Supplementary Note 6

The control device according to any one of Supplementary Notes 1 to 5,
wherein the processor is configured to:
   specify a system state for the CT scan; and
   acquire the scan parameter further based on the specified system state.

### Supplementary Note 7

A control method executed by at least one processor included in a control device used in a CT scan that acquires a plurality of pieces of projection data of a subject by a photon counting type radiation detector, the control method comprising:
specifying a scan region and an examination purpose of the subject;
acquiring a scan parameter based on the specified scan region and examination purpose; and
presenting the acquired scan parameter as part of a scan condition.

### Supplementary Note 8

A control processing program for causing at least one processor included in a control device used in a CT scan that acquires a plurality of pieces of projection data of a subject by a photon counting type radiation detector to execute processing comprising:
specifying a scab region and an examination purpose of the subject;
acquiring a scan parameter based on the specified scan region and examination purpose; and
presenting the acquired scan parameter as part of a scan condition.

### Supplementary Note 9

A computer program product including a control processing program for causing at least one processor included in a control device used in a CT scan that acquires a plurality of pieces of projection data of a subject by a photon counting type radiation detector to execute processing comprising:
specifying a scan region and an examination purpose of the subject;
acquiring a scan parameter based on the specified scan region and examination purpose; and
presenting the acquired scan parameter as part of a scan condition.

### Supplementary Note 10

A computer-readable storage medium storing a control processing program for causing at least one processor included in a control device used in a CT scan that acquires a plurality of pieces of projection data of a subject by a photon counting type radiation detector to execute processing comprising:
specifying a scan region and an examination purpose of the subject;
acquiring a scan parameter based on the specified scan region and examination purpose; and
presenting the acquired scan parameter as part of a scan condition.

What is claimed is:

1. A control device used in a CT scan that acquires a plurality of pieces of projection data of a subject by a photon counting type radiation detector, the control device comprising at least one processor configured to:
specify a scan region and an examination purpose of the subject;
acquire a scan parameter based on the specified scan region and examination purpose; and
present the acquired scan parameter as part of a scan condition.

2. The control device according to claim 1,
wherein the scan parameter is associated with at least one of the scan region or the examination purpose based on a rule.

3. The control device according to claim 2,
wherein the scan parameter includes a plurality of types, and
the processor is configured to acquire a first scan parameter in which a parameter value is associated based on a first rule corresponding to the scan region and a second scan parameter in which a parameter value is associated based on a second rule corresponding to the examination purpose.

4. The control apparatus according to claim 3,
wherein the processor is configured to present the second scan parameter as part of the scan condition, in a case where a type of the first scan parameter and a type of the second scan parameter are the same.

5. The control device according to claim 3,
wherein the second scan parameter includes at least one of a measurement mode or a focal size.

6. The control device according to claim 1,
wherein the processor is configured to:
specify a system state for the CT scan; and
acquire the scan parameter further based on the specified system state.

7. A control method executed by at least one processor included in a control device used in a CT scan that captures a plurality of pieces of projection data of a subject by a photon counting type radiation detector, the control method comprising:
specifying a scan region and an examination purpose of the subject;
acquiring a scan parameter based on the specified scan region and examination purpose; and
presenting the acquired scan parameter as part of a scan condition.

8. A non-transitory computer-readable storage medium storing a control processing program for causing at least one processor included in a control device used in a CT scan that acquires a plurality of pieces of projection data of a subject by a photon counting type radiation detector to execute processing comprising:
specifying a scan region and an examination purpose of the subject;
acquiring a scan parameter based on the specified scan region and examination purpose; and
presenting the acquired scan parameter as part of a scan condition.
